(19) United States
(12) Patent Application Publication
(10) Pub. No.: US 2018/0280064 A1
(43) Pub. Date: Oct. 4, 2018

(54) METHODS AND SYSTEMS FOR INCREASING THE BENDING STIFFNESS OF A SPINAL SEGMENT WITH ELONGATION LIMIT

(71) Applicant: EMPIRICAL SPINE, INC., WOODSIDE, CA (US)

(72) Inventors: Louis Fielding, San Carlos, CA (US); Ian Bennett, San Francisco, CA (US); Manish Kothari, San Carlos, CA (US); Todd Alamin, Woodside, CA (US); Hugues Malandain, Mountain View, CA (US); Craig Litherland, Palo Alto, CA (US); Colin Cahill, Portola Valley, CA (US)

(73) Assignee: EMPIRICAL SPINE, INC., WOODSIDE, CA (US)

(21) Appl. No.: 15/726,986

(22) Filed: Oct. 6, 2017

Related U.S. Application Data
(63) Continuation of application No. 13/073,620, filed on Mar. 28, 2011, now abandoned.
Application No. 13/073,620 is a continuation-in-part of application No. 12/535,560, filed on Aug. 4, 2009, now abandoned.
Application No. 12/535,560 is a continuation-in-part of application No. 12/106,103, filed on Apr. 18, 2008, now Pat. No. 8,403,961.
(60) Provisional application No. 60/936,897, filed on Jun. 22, 2007.

(51) Int. Cl. A61B 17/70

(52) U.S. Cl. CPC A61B 17/7067; A61B 17/7053

(57) ABSTRACT

A system for restricting spinal flexion includes a compliance member having a body and an elongation limit. The body typically comprises a spring or other tension element which provides elastic constraint to the spinal segment when the compliance member is attached to the spinous processes. The elongation limit prevents overextension of the compliance member, thus reducing the likelihood that the patient will experience over flexion of the spinal segment and reducing the risk of placing excessive mechanical load on the compliance member.

### CROSS-REFERENCES TO RELATED APPLICATIONS

This application is a continuation of U.S. patent application Ser. No. 13/073,620 (Attorney Docket No. 48626-705.502), filed Mar. 28, 2011, which is a continuation-in-part of U.S. patent application Ser. No. 12/535,560 (Attorney Docket No. 48626-705.501), filed on Aug. 4, 2009, (now abandoned), which is a continuation-in-part of U.S. patent application Ser. No. 12/106,103 (Attorney Docket No. 48626-705.201), filed on Apr. 18, 2008, (now U.S. Pat. No. 8,403,961), which claims the benefit of Provisional No. 60/936,897, (Attorney Docket No. 48626-705.101), filed on Jun. 22, 2007, the full disclosures of which are incorporated herein by reference.

The present invention is related to but does not claim priority from application Ser. No. 11/076,469, filed on Mar. 9, 2005, now U.S. Pat. No. 7,458,981, which claimed the benefit of prior provisional application 60/551,235, filed on Mar. 9, 2004; application Ser. No. 11/777,366 filed on Jul. 13, 2007; Application Ser. No. 11/827,980 filed on Jul. 13, 2007; PCT application no. US 2007/081815 filed on Oct. 18, 2007; PCT application no. US 2007/081822 filed on Oct. 18, 2007; and application Ser. No. 11/975,674 filed on Oct. 19, 2007, the full disclosures of which are incorporated herein by reference.

### BACKGROUND OF THE INVENTION

### 1. Field of the Invention

The present invention relates generally to medical methods and apparatus. More particularly, the present invention relates to methods and devices for restricting spinal flexion in patients having back pain or other spinal conditions.

A major source of chronic low back pain is discogenic pain, also known as internal disc disruption. Patients suffering from discogenic pain tend to be young, otherwise healthy individuals who present with pain localized to the back. Discogenic pain usually occurs at the lower lumbar discs of the spine (FIGS. 1 and 1A). Pain is typically exacerbated when patients put their lumbar spines into flexion (i.e. by sitting or bending forward) and relieved when they put their lumbar spines into extension (i.e. the standing position, or arching backwards). Discogenic pain can be quite disabling, and for some patients, can dramatically affect their ability to work and otherwise enjoy their lives.

Such discogenic low back pain can be thought of as flexion instability and is related to flexion instability that is manifested in other conditions. The most prevalent of these is spondylolisthesis, a spinal condition in which abnormal segmental translation is exacerbated by segmental flexion.

Current treatment alternatives for patients diagnosed with chronic discogenic pain are quite limited. Many patients follow a conservative treatment path, such as physical therapy, massage, anti-inflammatory and analgesic medications, muscle relaxants, and epidural steroid injections, but typically continue to suffer with a significant degree of pain. Other patients elect to undergo spinal fusion surgery, which commonly requires discectomy (removal of the disk) together with fusion of adjacent vertebrae. Fusion is not usually recommended for discogenic pain because it is irreversible, costly, associated with high morbidity, and of questionable effectiveness. Despite its drawbacks, however, spinal fusion for discogenic pain remains common due to the lack of viable alternatives.

An alternative method, that is not commonly used in practice, but has been approved for use by the FDA, is the application of bone cerclage devices that can encircle the spinous processes or other vertebral elements and thereby create a restraint to motion. Physicians typically apply a tension or elongation to the devices that applies a constant and high force on the anatomy, thereby fixing the segment in one position and allowing effectively no motion. The lack of motion allowed after the application of such a device is thought useful to improve the likelihood of fusion performed concomitantly; if the fusion does not take, these devices will fail through breakage of the device or of the spinous process to which the device is attached. These devices are designed for static applications and are not designed to allow for a dynamic elastic resistance to flexion across a range of motion. The purpose of bone cerclage devices and the other techniques described above is to almost completely restrict measurable motion of the vertebral segment of interest. This loss of motion at a given segment gives rise to abnormal loading and motion at adjacent segments, leading eventually to adjacent segment morbidity.

Recently, a less invasive and potentially more effective treatment for discogenic pain has been proposed. A spinal implant has been designed which inhibits spinal flexion while allowing substantially unrestricted spinal extension. The implant is placed over one or more adjacent pairs of spinous processes and provides an elastic restraint to the spreading apart of the spinous processes which occurs during flexion. Such devices and methods for their use are described in U.S. Pat. No. 7,458,981, which has common inventors with the present application.

Implants used for applying elastic constraint to spinal segments as taught in the '981 patent must meet two generally conflicting objectives. First, the implants must be very robust and have a high fatigue strength since they will be subjected to use over millions of cycles after implantation as the patient goes about daily life. For a given desired stiffness, the principle way in which to increase fatigue strength is to increase the size of the implant. Generally, however, smaller implants having a lower profile are easier to implant, are better tolerated by the patient, and lead to fewer complications.

In addition to the limitations on size and strength, as discussed above, implantable elastic constraints as described in the '981 patent can, in some cases, allow excess flexion despite the elastic constraint which is applied as the spinous processes of the spinal segment move apart. Should such excess flexion occur, the patient can experience pain and the implant itself can experience greater stress and fatigue than intended.

For these reasons, it would be desirable to provide improved spinal implants and methods for their use in inhibiting flexion in patients suffering discogenic pain. It would be particularly desirable if such improved devices would be robust in use with very high fatigue strengths while having a minimum size and correspondingly reduced implantation profile. It would be further desirable if, in addition to the strength and size characteristics, the elastic constraints were to inhibit or prevent excess flexion of the treated spinal segment during use. At least some of these objectives will be met by the invention as described herein below.

### 2. Description of the Background Art

U.S. Pat. No. 7,458,981 has been described above. US 2005/0192581 describes an orthopedic tether which can have a stiffness from at least 1 N/mm to at least 200 N/mm and which can be used for many purposes, including wrapping spinous processes. U.S. 2008/0312693 describes a spine stabilization unit comprising a spring with an internal motion limit. Other patents and published applications of interest include: U.S. Pat. Nos. 3,648,691; 4,643,178; 4,743,260; 4,966,600; 5,011,494; 5,092,866; 5,116,340; 5,180,393; 5,282,863; 5,395,374; 5,415,658; 5,415,661; 5,449,361; 5,456,722; 5,462,542; 5,496,318; 5,540,698; 5,562,737; 5,609,634; 5,628,756; 5,645,599; 5,725,582; 5,902,305; Re. 36,221; 5,928,232; 5,935,133; 5,964,769; 5,989,256; 6,053,921; 6,248,106; 6,312,431; 6,364,883; 6,378,289; 6,391,030; 6,468,309; 6,436,099; 6,451,019; 6,582,433; 6,605,091; 6,626,944; 6,629,975; 6,652,527; 6,652,585; 6,656,185; 6,669,729; 6,682,533; 6,689,140; 6,712,819; 6,689,168; 6,695,852; 6,716,245; 6,761,720; 6,835,205; 7,029,475; 7,163,558; Published U.S. Patent Application Nos. US 2002/0151978; US 2004/0024458; US 2004/0106995; US 2004/0116927; US 2004/0117017; US 2004/0127989; US 2004/0172132; US 2004/0243239; US 2005/0033435; US 2005/0049708; US 2006/0069447; US 2006/0136060; US 2006/0240533; US 2007/0213829; US 2007/0233096; Published PCT Application Nos. WO 01/28442 A1; WO 02/03882 A2; WO 02/051326 A1; WO 02/071960 A1; WO 03/045262 A1; WO 2004/052246 A1; WO 2004/073532 A1; and Published Foreign Application Nos. EP 0322334 A1; and FR 2 681 525 A1. The mechanical properties of flexible constraints applied to spinal segments are described in Papp et al. (1997) Spine 22:151-155; Dickman et al. (1997) Spine 22:596-604; and Garner et al. (2002) Eur. Spine J. S186-S191; Al Baz et al. (1995) Spine 20, No. 11, 1241-1244; Heller, (1997) Arch. Orthopedic and Trauma Surgery, 117, No. 1-2:96-99; Leahy et al. (2000) Proc. Inst. Mech. Eng. Part H: J. Eng. Med. 214, No. 5: 489-495; Minns et al., (1997) Spine 22 No. 16:1819-1825; Miyasaka et al. (2000) Spine 25, No. 6: 732-737; Shepherd et al. (2000) Spine 25, No. 3: 319-323; Shepherd (2001) Medical Eng. Phys. 23, No. 2: 135-141; and Voydeville et al (1992) Orthop Traumatol 2:259-264.

### SUMMARY OF THE INVENTION

The present invention provides methods and apparatus for relieving symptoms of lumbar pain associated with flexion of a spinal segment of a patient. The lumbar pain may arise from a variety of particular conditions such as those described previously herein. The devices and methods will dynamically limit flexion of at least one spine segment by increasing the bending stiffness of the spinal segment by a preselected amount, typically in the range from 0.1 Nm/deg to 2 Nm/deg, preferably from 0.4 Nm/deg to 1 Nm/deg. Usually, the bending stiffness is increased by coupling an elastic constraint between a superior spinous process and an inferior spinous process or between an L5 spinous process and a sacrum of the patient. The elastic constraint may have an effective elastic tensile stiffness in the range from 7.5 N/mm to 40 N/mm, where the constraint may be positioned at a distance in the range from 25 mm to 75 mm in a posterior direction from a center of rotation of the spinal segment. The “effective elastic tensile stiffness” is defined as the elastic tensile stiffness present between the inferior and superior attachment location resulting from the stiffness contributions of all elements or components of the elastic constraint. The bending stiffness will be increased during flexion (but not extension) of the spinal segment, usually being increased over the full range of flexion. The full flexion-extension range of motion of the spinal segment will typically be from 3.degree. to 20.degree., usually from 5.degree. to 15.degree. The flexion portion of the total range of motion of the spinal segment is expressed as an angle measured relative to the neutral position (defined below) and will typically be from 2.degree. to 15.degree., usually from 4.degree. to 10.degree. The bending stiffness will be increased over at least 75% of the full range flexion, usually over the full range of flexion as well as 25% of the extension range of motion.

In addition to dynamically limiting flexion of the at least one spine segment by increasing the segment's bending stiffness, the devices and methods of the present invention further provide for a “hard” limit or stop on flexion to both reduce the risk of the patient suffering from over flexion of the spinal segment and to reduce the mechanical load on the elastic constraint devices. Usually, the flexion limit is provided by a separate elongation limit or limiting element which is flexible but substantially non-distensible and which may be attached between vertically adjacent spinous processes (or an L5 process and a sacrum) in parallel with the elastic constraint. “Substantially non-distensible”as used in this application is defined as having a higher tensile stiffness than the compliance element; usually having a tensile stiffness which is at least twice that of the compliance element, and preferably having a tensile stiffness which is at least ten times that of the compliance element. The elongation limiting element will be attached so that there is some slack or excess length present when the spinal segment is in its neutral position. Thus, as the spinal segment initially undergoes flexion, the elastic constraint will provide the desired increase in bending stiffness while the elongation limit applies little or no force between the spinous processes. Once the spinal segment reaches the desired maximum flexion, however, the elongation limit will reach its maximum extension and prevent further separation of the spinous processes, thus protecting both the patient from over flexion and the elastic constraint from excess stress.

The elongation limit may have any one of a variety of configurations, but will usually comprise a tether, cord, or cable made from a very flexible but substantially non-distensible (i.e. very high tensile stiffness) material (as defined above) which prevents further separation of the spinous processes as soon as it goes taut. In an exemplary embodiment, the tether may consist of a single cord, e.g., formed from ultra high molecular weight polyethelene fibers, braids, cords, tubes, or the like. In alternative embodiments, the elongation limit may be incorporated into the elastic constraint so that the elongation limit is in its slack state when the elastic constraint is in its neutral position between the spinous processes of the spinal segment.

The preferred methods and systems of the present invention will provide minimum and preferably no elastic resistance to extension of the spinal segments. The preferred elastic constraint systems of the present invention will be coupled to the spinous processes via flexible straps which, by virtue of their placement around the spinous processes and their flexible nature, will impart no force to the spinous processes as they move together during extension. Furthermore, the implants of the present invention will usually be free from structure located between adjacent spinous processes, although in some cases structure may be provided where the structure does not substantially interfere with or impede the convergence of the spinous processes as the spine undergoes extension. While some small amount of elastic resistance to extension might be found, it will preferably be below 3 N/mm, more preferably below 1 N/mm, and usually below 0.5 N/mm.

Similarly, the preferred methods and systems of the present invention will provide a minimum and preferably no elastic resistance to lateral bending or rotation of the spinal segments. The preferred methods and systems of the present invention will usually be coupled to the spinous processes via flexible straps which, by virtue of their placement around the spinous processes and their flexible nature, make it very difficult for the preferred methods and systems of the present invention to provide any resistance to lateral bending or rotation. This is particularly true in the lumbar spine where the range of motion in rotation is usually limited to .sup . . . +−0.3.degree. While some small amount of elastic resistance to lateral bending or rotation might be found, it will preferably be small.

As used herein, the phrase “spinal segment” refers to the smallest physiological motion unit of the spine which exhibits mechanical characteristics similar to those of the entire spine. The spinal segment, also referred to as a “functional spinal unit” (FSU), consists of two adjacent vertebrae, the intervertebral disk, and all adjoining ligaments and tissues between them. For a more complete description of the spinal segment or FSU, see White and Panjabi, Clinical Biomechanics of the Spine, J.B. Lippincott, Philadelphia, 1990.

As used herein, “neutral position” refers to the position in which the patient's spine rests in a relaxed standing position. The “neutral position” will vary from patient to patient. Usually, such a neutral position will be characterized by a slight curvature or lordosis of the lumbar spine where the spine has a slight anterior convexity and slight posterior concavity. In some cases, the presence of the constraint of the present invention may modify the neutral position, e.g. the device may apply an initial force which defines a new neutral position having some small extension of the untreated spine. As such, the use of the term “neutral position” is to be taken in context of the presence or absence of the device. As used herein, “neutral position of the spinal segment” refers to the position of a spinal segment when the spine is in the neutral position.

As used herein, “segmental flexion” refers to the motion between adjacent vertebrae in a spinal segment as the patient bends forward. Referring to FIG. 1A, as a patient bends forward from the neutral position of the spine, i.e. to the right relative to a curved axis A, the distance between individual vertebrae L on the anterior side decreases so that the anterior portion of the intervertebral disks D are compressed. In contrast, the individual spinous processes SP on the posterior side move apart in the direction indicated by arrow B. Segmental flexion thus refers to the relative movement between adjacent vertebrae as the patient bends forward from the neutral position illustrated in FIG. 1A.

As used herein, “segmental extension” refers to the motion of the individual vertebrae L as the patient bends backward and the spine extends from the neutral position illustrated in FIG. 1A. As the patient bends backward, the anterior ends of the individual vertebrae will move apart. The individual spinous processes SP on adjacent vertebrae will move closer together in a direction opposite to that indicated by arrow B.

As used herein, the phrases “elastic resistance” and “elastic stiffness” refer to an application of constraining force to resist motion between successive, usually adjacent, spinous processes such that increased motion of the spinous processes results in a greater constraining force. The elastic resistance or stiffness will, in the inventions described herein, inhibit motion of individual spinal segments by, upon deformation, generating a constraining force transmitted directly to the spinous processes or to one or more spinous process and the sacrum. The elastic resistance or stiffness can be described in units of stiffness, usually in units of force per deflection such as Newtons per millimeter (N/mm). The stiffness may be defined for a single component or compliance member or for the entire structure which may comprise more than one compliance member. In some cases, the elastic resistance will generally be constant (within .+−0.5%) over the expected range of motion of the spinous processes or spinous process and sacrum. In other cases, typically with elastomeric components as discussed below, the elastic resistance may be non-linear, potentially varying from 33% to 100% of the initial resistance over the physiologic range of motion. Usually, in the inventions described herein, the pre-operative range of motion of the spinous process spreading from the neutral or upright position to a maximum flexion-bending position will be in the range from 2 mm to 20 mm, typically from 4 mm to 12 mm. With the device implanted, the post-operative range of motion of the spinous process spreading from the neutral or upright position to a maximum flexion-bending position will be reduced and will usually be in the range from 1 mm to 10 mm, typically from 2 mm to 5 mm. Such spinous process spreading causes the device to undergo deformations of similar magnitude.

As used herein, the phrase “bending stiffness” is defined as the resistance of the spinal segment to bending. The incremental bending stiffness which is provided by the constraints of the present invention may be calculated based on the total elastic tensile stiffness (or elastic resistance) of the constraint circumscribing the spinous processes (or coupling the L5 spinous process to sacrum) and the distance or moment arm between a center of rotation (COR) of the spinal segment and the location at which the elastic constraint is located on the spinous processes. As used herein, the moment arm distance will be expressed in meters (m) and the elastic stiffness ES will be expressed in Newtons per millimeter (N/mm). In the preferred embodiments where two compliance members are positioned “in parallel,” the total elastic stiffness of the constraint structure will be twice the elastic stiffness of a single compliance member. The units of bending stiffness, as used herein, will be Newton-meters per degree (Nm/deg.). The increase in bending stiffness IBS provided by the constraint of the present invention can be calculated by the formula:

IBS=1000 ES×D²×(π/180°)

where the elastic stiffness ES of the device can be measured by testing the device on an Instron® or other tensile strength tester, and the moment arm length D can be measured from radiographs.

Alternatively, the increase in bending stiffness of a device could be measured directly by placement on a cadaveric spine segment or a suitable vertebral model. The bending stiffness of the spine segment could be measured with and without the elastic constraint and the increase in bending stiffness provided by the constraint would be the difference between the two values. It would also be possible to calculate the increase in bending stiffness by finite element analysis.

The bending stiffness increase can thus be adjusted by changing the tensile stiffness of the elastic constraint and/or the distance of the moment arm. For example, once the treating physician determines the location of the elastic constraint and the distance between that location and the center of rotation (COR), the physician can then choose an elastic constraint having an appropriate elastic tensile stiffness in order to achieve a target therapeutic increase in the bending stiffness. The location of the center of rotation and the distance of the moment arm can be determined from radiographic images of the target spinal segment, typically taken in at least two positions or postures, such as in flexion and in extension. Typically, the center of rotation will be an average or calculated value determined by measuring translational vectors between the two radiographic positions for two points on a vertebra. Such techniques are described in detail, for example, in Musculoskeletal Biomechanics. Paul Brinckmann, Wolfgang Frobin, Gunnar Leivseth (Eds.), Georg Thieme Verlag, Stuttgart, 2002; p. 105. It would also be possible to employ the instantaneous axis of rotation (IAR), which location varies depending on the degree of spinal flexion or extension. Generally, however, using the COR is preferred since it is a fixed and readily determined value, although the device may affect the location of the COR in some cases.

Thus, the bending stiffness applied by a constraining structure according to the present invention is increased when the spinal segment moves beyond the neutral position and will depend on several factors including the elastic characteristics of the constraining structure, the position of the constraining structure on the spinous processes, the dimensions of the constraining structure, and the patient's anatomy and movement. The constraining structure will usually be positioned so that the upper and lower tethers engage the middle anterior region of the spinous process (25 mm to 75 mm posterior of the COR), and the dimensions of the constraining structure will usually be adjusted so that the tethers are taut, i.e. free from slack, but essentially free from tension (axial load) when the spinal segment is in its neutral position, i.e., free from flexion and extension. As the segment flexes beyond the neutral position, the constraining structure will immediately provide an elastic resistance in the ranges set forth above.

In some cases, the dimensions and assembly of the construct will be selected so that the tethers and compliance members are slightly pre-tensioned even before the compliance members are under load. Thus, the constraining structure may apply a predetermined resistive force, typically in the range from 7.5 N to 40 N, as soon as the spinal segment flexes from the neutral position. In the absence of such pre-tensioning, the compliance members would apply a zero resistive force at the instant they are placed under load. In all cases, as the segment flexes beyond the treated neutral position, the constraining structure will provide increasing bending stiffness in the ranges set forth above.

Usually, the constraining structures will apply minimal or no bending stiffness when the spinal segment is in the neutral position. In some instances, however, it may be desirable to tighten the constraining structure over the spinous processes so that a relatively low finite bending stiffness force (typically in the range from 0.1 Nm/deg to 2 Nm/deg, usually from 0.4 Nm/deg to 1 Nm/deg) is applied even before flexion while the spinal segment remains at a neutral position. In this case, the additional stiffness afforded by the constraining structure will affect the entire flexion range of motion; as well as a portion of the untreated extension range of motion of the spinal segment.

The relative increase in bending stiffness afforded by the constraining structures of the present invention is advantageous because it allows the constraining structure to cause the treated segment to resist flexion sufficiently to relieve the underlying pain or instability with a reduced risk of injury from excessive force. In particular, the preferred bending stiffness ranges set forth above provide sufficient constraint to effect a significant change in flexion in the typical patient while allowing a significant safety margin to avoid the risk of injury. The bending stiffness provided by the constraints of the present invention will limit the separation of the spinous processes on the treated spinal segment which is desirable both to reduce flexion-related pain and spinal instability.

The resistance to flexion provided by the elastic constraints of the present invention may reduce the angular range-of-motion (ROM) relative to the angular ROM in the absence of constraint. Angular ROM is the change in angle between the inferior end plate of the superior vertebral body of the treated segment and the superior endplate of the inferior vertebral body of the treated segment when the segment undergoes flexion. Thus, the treatments afforded by the elastic constraints of the present invention will provide a relatively low angular ROM for the treated segment, but typically a ROM higher than that of a fused segment.

While the constraint structures of the present invention will limit flexion, it is equally important to note that in contrast to spinal fusion and immobilizing spinal spacers, the methods and devices of the present invention will allow a controlled degree of flexion to take place. Typically, the methods and devices of the present invention will allow a degree of flexion which is equal to at least about 20% of the flexion that would be observed in the absence of constraint, more typically being at least about 33%. By reducing but not eliminating flexion, problems associated with fusion, such as increased pain, vertebral degeneration, instability at adjacent segments, and the like, may be overcome.

The constraint structures of the present invention will act to restore the stiffness of a spinal segment which is “lax” relative to adjacent segments. Often a patient with flexion-related pain or instability suffers from a particular looseness or laxity at the painful segment. When the patient bends forward or sits down, the painful, lax segment will preferentially flex relative to the stiffer adjacent segments. By adjusting the length, position, or other feature of the devices of the present invention so that constraint structure is taut over the spinous processes when the spinal segment is in its neutral position, the stiffness of the treated segment can be “normalized” immediately as the patient begins to impart flexion to the spine. Thus, premature and/or excessive flexion of the target spinal segment can be inhibited or eliminated.

The protocols and apparatus of the present invention allow for individualization of treatment. Compliance members with different stiffnesses, elongations (lengths of travel), placement location in the anterior posterior direction along the spinous processes and other characteristics can be selected for particular patients based on their condition. For example, patients suffering from a severe loss of stiffness in the target spinal segment(s) may be treated with devices that provide more elastic resistance. Conversely, patients with only a minimal loss of natural segmental stiffness can be treated with devices that provide less elastic resistance. Similarly, bigger patients may benefit from compliance members having a greater maximum elongation, while smaller patients may benefit from compliance members having a shorter maximum elongation.

For some patients, particularly those having spinal segments which are very lax, having lost most or all of their natural segmental stiffness, the present invention can provide for “pre-tensioning” of the constraining structure. As described above, one way to accomplish this is by shortening the constraining structure such that a small amount of tension is held by the constraining structure when the spine is in the neutral or slightly extended initial position. Alternatively, pre-tensioned compliance elements can be provided to pre-tension the constraining structure without changing its length. The tension or compression elements utilized in the compliance members of the present invention, such as coil springs, elastomeric bodies, and the like, will typically present little or no elastic resistance when they are first deformed. Thus, there will be some degree of elongation of the compliance members prior to the spinal segment receiving a therapeutic resistance. To provide a more immediate relief, the tension or compression members may be pre-tensioned to have an initial static resistive force which must be overcome to initiate deformation. In this way, a constrained spinal segment will not begin to flex at the instant the patient begins to flex her or his spine which is an advantage when treating lax spinal segments. Certain specific embodiments for achieving such pre-tensioning are described in detail below.

In a first specific aspect of the present invention, a compliance member for attaching inelastic tethers circumscribing spinal processes comprises a body and an elongation limit. The body has a superior tether attachment element and an inferior tether attachment element, and the body defines a tension spring capable of elastic elongation when said attachment elements are drawn apart. The elongation limit is coupled between the superior tether attachment and the inferior tether attachment to prevent elongation of the tension spring beyond a maximum elongation length. Typically, the maximum elongation length is in the range from 2 mm to 15 mm, usually from 5 mm to 10 mm. The constraint structure typically has a total elastic stiffness in the range from 7.5 N/mm to 40 N/mm; and thus a single compliance member in the preferred parallel configuration typically has an elastic stiffness in the range from 3.75 N/mm to 20 N/mm. The compliance member may comprise a variety of elements or components which are able to be attached between the tether attachments of the compliance member body, typically being a non-distensible tether, such as a braided cord with a tensile stiffness greater than 20 N/mm, preferably being greater than 100 N/mm. The non-distensible tether may be secured over an exterior of the body of the compliance member, for example in the form of a braided jacket, tube or sheath. More usually, however, the non-distensible tether will be secured within an interior of the body of the compliance member, for example consisting of a single cord extending from the inferior tether attachment to the superior tether attachment or comprising two or more cords secured between the inferior and superior tether attachments. In a specific embodiment, the tether may be part of an assembly including a base, where the base is secured near one of the tether attachment and the cord looped around an anchor secured near the other of the tether attachments.

The compliance members of the present invention will typically be incorporated into a system for elastically constraining flexion of a spinal segment. Such systems will comprise first and second compliance members, a first non-distensible tether adapted to attach to the first tether attachment element of the first compliance member and to the second tether attachment element of the second compliance member, a second non-distensible tether adapted to attach to the first tether attachment element of the second compliance and to the second tether attachment element of the first compliance member.

In a second specific aspect of the present invention, methods for relieving symptoms of lumbar pain associated with spinal segment flexion comprise coupling and elastic constraint between a superior spinous process and an inferior spinous process or sacrum of the spinal segment. The elastic constraint increases the bending stiffness of the spinal segment in flexion sufficiently to reduce lumbar pain. Of particular interest to the present invention, elongation of the elastic restraint is limited to a maximum elongation length to prevent excessive flexion of the spinal segment, both decreasing the risk of patient discomfort resulting from over flexion of the segment and reducing the maximum stress experienced by the elastic constraint.

Usually, the maximum elongation length for the elastic restraint is in the range from 2 mm to 15 mm, more usually being between 5 mm and 10 mm, with respect to the neutral position of the spinal segment. Limiting elongation typically comprising coupling an inelastic constraint between the superior spinous process and the inferior spinous process, where the inelastic constraint when fully extended is longer than the elastic constraint when coupled to the spinous processes of the spinal segment in a neutral position by a length equal to the maximum elongation length.

Elastic constraint typically increases the bending stiffness of the spinal segment by an amount in the range from 0.1 Nm/deg to 2 Nm/deg. In particular, the elastic constraint may have a total elastic stiffness in the range from 7.5 N/mm to 40 N/mm when the constraint is positioned at a distance in the range from 25 millimeters to 75 millimeters in a posterior direction from a center of rotation of the spinal segment. Optionally, the methods may further comprise adjusting the elastic constraint so that it is taut but not stretched over the spinous processes or L5 spinous process and sacrum when the spinal segment is in its neutral position. The methods may further comprise changing the length of the elastic constraint after it has been coupled to the spinous processes or L5 spinous process and sacrum. Optionally, the bending stiffness may be increased over at least a portion of the full flexion range of the motion of the spinal segment, usually being increased over the entire full flexion range of motion of the spinal segment.

### BRIEF DESCRIPTION OF THE DRAWINGS

FIG. 1 is a schematic diagram illustrating the lumbar region of the spine including the spinous processes (SP), facet joints (FJ), lamina (L), transverse processes (TP), and sacrum (S).

FIG. 1A is a schematic illustration illustrating a portion of the lumbar region of the spine taken along a saggital plane.

FIGS. 1B and 1C illustrate a spinal segment having a center of rotation (COR) both in a neutral position (FIG. 1B) and in a fully flexed position (FIG. 1C).

FIG. 2 is a schematic illustration of the systems of the present invention comprising superior and inferior tether structures and right and left compliance members.

FIG. 3 illustrates an exemplary coil spring tension member.

FIG. 3A illustrates the coil spring tension member of FIG. 4 illustrating the preferred dimensions.

FIGS. 4A-4C illustrate the use of a locking mechanism incorporated in the tension member of FIG. 3 for removably securing a band member of a tether structure.

FIGS. 5A and 5B illustrate a constraint assembly similar to that shown in FIGS. 10A and 10B where the sheath contains elements which minimize sheath interaction with the tension element and/or limit the maximum elongation of the assembly under tension.

FIGS. 6A and 6B illustrate an accordion-type sheath which could potentially also limit maximum elongation.

FIGS. 7A-7D illustrate different embodiments of internal tethers used to provide the elongation limits of the present invention.

FIGS. 8A and 8B illustrate external tethers used to provide the elongation limits of the present invention.

### DETAILED DESCRIPTION OF THE INVENTION

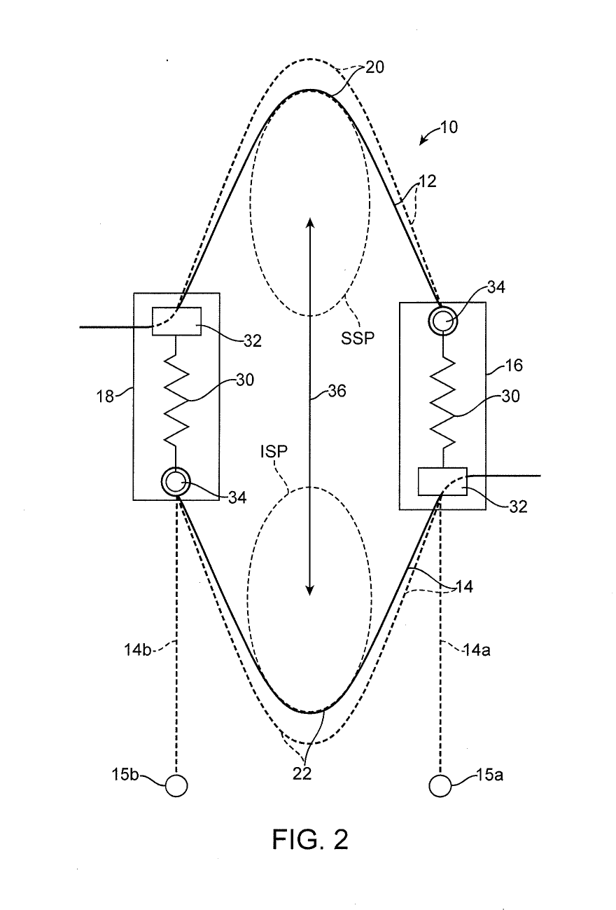

Exemplary spinous process constraints according to the present invention are illustrated schematically in FIG. 2. The systems 10 comprise a superior tether structure 12, and inferior tether structure 14, and right compliance member 16 and a left compliance member 18. The superior tether structure 12 will typically be a continuous band, cable, strap, cord, or other structure which extends between the two compliance members and provides a saddle region 20 which is adapted to lie over and conform to a superior surface of a superior spinous process SSP as described in more detail in the related prior applications which have been incorporated herein by reference. The inferior tether structure 14 will typically comprise a band, cable, or the like which is constructed similarly if not identically to the superior tether structure 12 and has a saddle region 22 adapted to lie over and conform to an inferior surface of an inferior spinous process 22. In certain instances, however, the inferior tether structure 14 may comprise separate bands, cables, straps, cords, or the like, 14a and 14b, shown in broken line, which have anchors 15a and 15b at their lower ends and are adapted to be separately attached to an inferior vertebrae or more commonly to a sacrum. The use of such separate tether structures for inferior attachment are described in more detail in co-pending application Ser. No. 11/827,980 (Attorney Docket No. 026398-000120US), the full disclosure of which has been previously incorporated herein by reference. The tether structures will usually be flexible but effectively non-distensible so that they allow minimum elongation under tensile load.

The right and left compliance members 16 and 18 will usually have similar or identical constructions and include an adjustable attachment component 32 and a fixed attachment component 34 for securing connecting segments of the superior and inferior tether structures 12 and 14. Usually, each compliance member 16 and 18 will have one of the tether structures 12 and 14 pre-attached to the fixed attachment component 34. The two subassemblies can then be introduced onto opposite sides of the spinous processes, and the tether structures placed over the spinous processes or otherwise attached to the vertebral bodies, as generally described in co-pending application Ser. No. 11/875,674 (Attorney Docket No. 026398-000150US), the full disclosure of which is incorporated herein by reference.

The present invention is particularly concerned with the nature of the tension elements 30, and a number of specific embodiments will be described hereinbelow. In general, the tension elements 30 will elastically elongate as tension is applied by the superior and inferior tether structures 12 and 14 through the attachments 32 and 34, in the direction shown by arrow 36. As the spinous processes or spinous process and sacrum move apart during flexion of the constrained spinal segment, the superior and inferior tether structures 12 and 14 will also move apart, as shown generally in broken line in FIG. 2. A tension element 30 will elastically resist the spreading with a force determined by the mechanical properties of the tension member. In particular, the tension members will be selected to have a tensile or elastic stiffness, also known as a spring constant, in the relatively low ranges set forth above. Such low elastic constricting forces provide a number of advantages when compared to complete restriction or constriction with a high elastic force as described above.

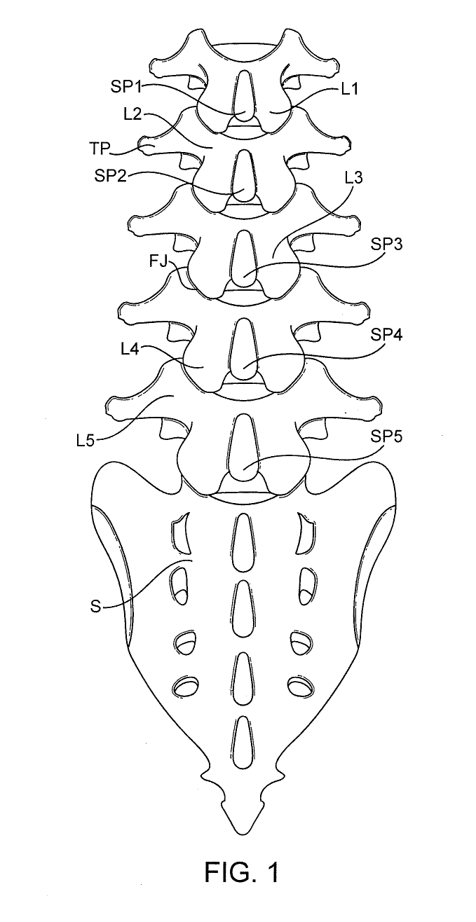

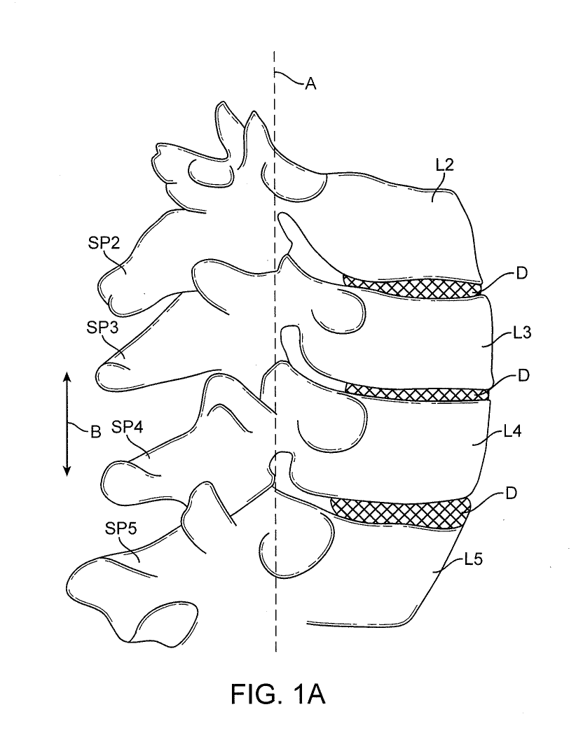

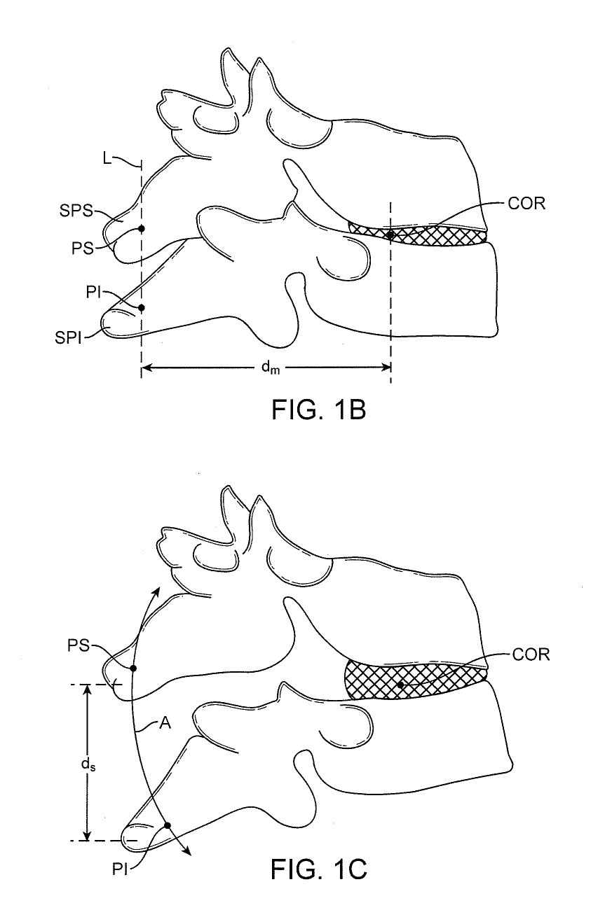

The tension elements of the present invention will be positioned over adjacent spinous processes, or over the L5 spinous process and adjacent sacrum, in order to increase the bending stiffness of the spinal segment. Referring to FIGS. 1B and 1C, the bending resistance is the resistance to bending of the spinal segment about a center of rotation (COR) positioned generally within or adjacent to the disk between adjacent vertebral bodies. The center of rotation can be determined from radiographic images, generally as described above, and it can be seen that a point PS on the superior spinous process SPS and a similar point PI on the inferior spinous process SPI will move generally along a curved line or arc A as shown in FIG. 1C. While the center of rotation COR is not fixed during flexion or extension of the spinal segment, and the points will not travel on a true arc, the motion of the spinous processes is nonetheless arcuate in nature as illustrated.

Thus, the positioning of any of the elastic constraints as described herein at a position on the spinous processes SPS and SPI generally indicated by line L will define a moment arm distance dₘ, as illustrated in FIG. 1B. The position L will generally be selected so that the moment arm length d.sub.m will be in the range from 25 mm to 75 mm, preferably from 40 mm to 60 mm. By thus selecting an elastic constraint having a total stiffness in the range from 7.5 N/mm to 40 N/mm, the desired bending stiffness of the spinal segment can be increased by an amount in the range from 0.1 Nm/deg to 2 Nm/deg, preferably from 0.4 Nm/deg to 1 Nm/deg.

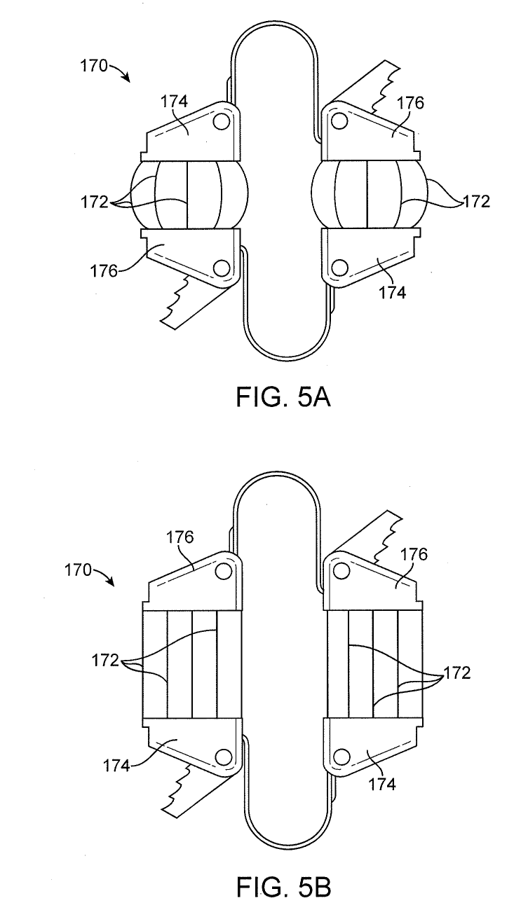

As also shown on FIG. 1C, the spinous processes SPS and SPI will spread to a maximum distance d.sub.s upon full flexion of the spinal segment. In accordance with other aspects of the present invention, it may be desirable to constrain the spreading of the spinous processes to a maximum distance above the distance in the neutral position (as shown in FIG. 1B) in the range from 1 mm to 10 mm, preferably from 2 mm to 8 mm. Certain of the elastic constraints in the present invention can provide for both increased bending stiffness and for a complete stop of flexion. See, for example, the device described in FIGS. 5A and 5B hereinafter.

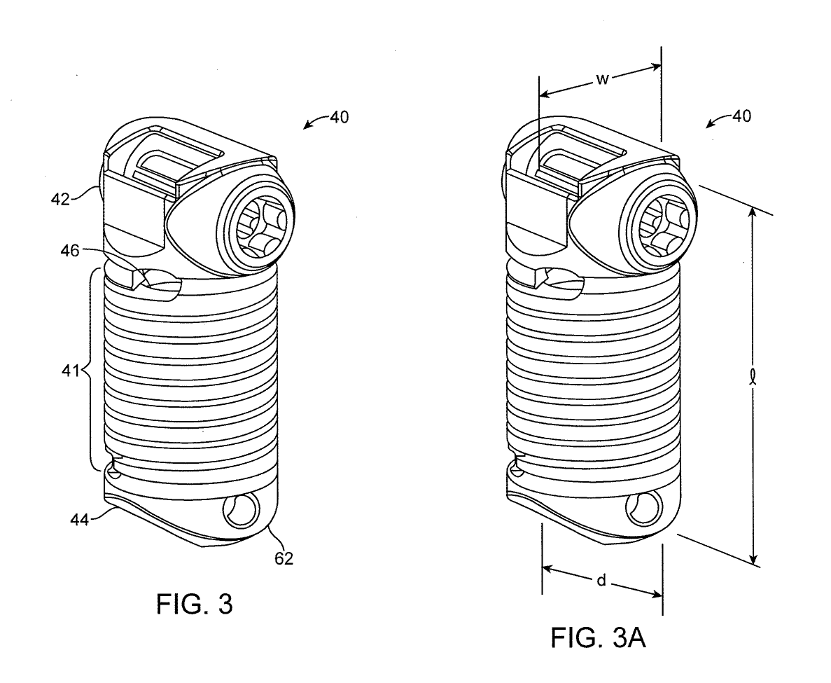

A first exemplary tension element 40 constructed in accordance with the principles of the present invention is illustrated in FIG. 3. The tension element 40 comprises a helical spring structure 41 formed from a single piece of material. The tension member 40 includes an adjustable tether connector 42 and a fixed tether connector 44, both of which are preferably formed integrally or monolithically with the helical spring structure 41. Typically, the helical spring structure 41 and both tether connectors 42 and 44 will be formed from one piece of material, usually being a metal such as titanium, but optionally being a polymer, ceramic, reinforced glass or other composite, or other material having desired elastic and mechanical properties and capable of being formed into the desired geometry. In a preferred embodiment, the tension member 40 is machined or laser cut from a titanium rod. Alternatively, a suitable polymeric material will be polyethylene ether ketone (PEEK). Other features may be built into the tension member 40, such as a stress relief hole 46. Components that mate with the adjustable tether connector may potentially include a roller and a lock-nut; such components could be made from the same material as the tension element and adjustable tether connector (e.g. titanium components if the tension member is titanium), or they could be made from a different material (e.g. injection molded PEEK).

Referring now to FIG. 3A, preferred dimensions for the tension member 40 are illustrated. In order to accommodate the patient anatomy when the tension members are arranged laterally of and vertically between adjacent spinous processes, as generally shown in FIG. 2, the compliance member will have a length 1 of 38 mm or less, preferably in the range from 20 mm to 30 mm, a depth d in the anterior-posterior direction no greater than 18 mm, preferably in the range from 8 mm to 15 mm, and a width in the direction normal to depth no greater than 15 mm, preferably in the range from 7 mm to 10 mm.

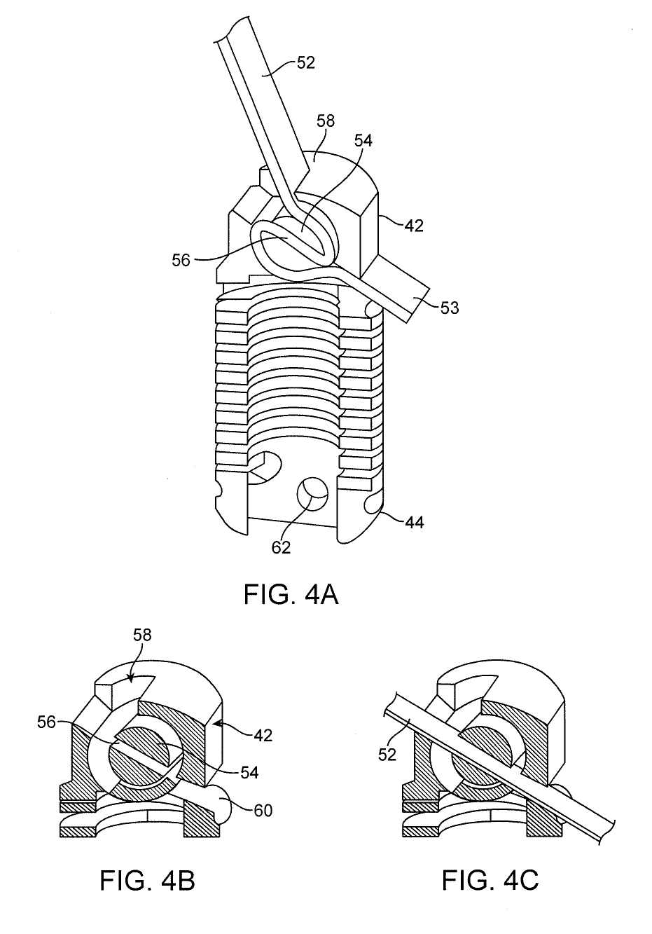

A free end 53 of the tether structure 52 may be attached to the adjustable tether connector 42, as illustrated in FIG. 4A through 4C. Initially, a barrel locking mechanism 54 is rotationally aligned such that a slot 56 is aligned with an inlet opening 58 on the top of the connector 42 and an outlet opening 60 on the side of the connector. The inlet opening 58 is located centrally and provides a primarily axial load on the compliance member, thereby evenly loading the compliance member and having the advantages described above. The free end 53 of tether 52 is then advanced through the inlet opening 58, slot 56, and outlet opening 60, as illustrated in FIG. 4C. By then rotating the barrel lock 54 approximately 180.degree., the tether 52 will be locked in place in the connector 42, as shown in FIG. 4A. It will be appreciated that this simple locking mechanism allows tether 52 to be appropriately tensioned for the individual patient before locking the tether in place. A locking feature, e.g. set screw, nut, or pin (not shown) would then be used to lock the tether and roller in place, providing additional resistance to unfurling and opening. The tensioning could be performed separately and/or simultaneously during implantation of the constraint assembly. Additional features of the mechanism such as pins, shoulders, or other features which control the travel of the roller or lock-nut may aid in the alignment and operation of the mechanism.

Another tether structure (not illustrated) will be attached to the fixed connector 44 at the other end of the tension element 40, typically using a pin (not illustrated). The pin may be anchored in a pair of receiving holes 62, and a free end of the tether wrapped over the pin and firmly attached. Usually, the fixed tether structure will be pre-attached at the time of manufacture so that the treating physician can implant each of the pair of tension members, with one tether structure attached to the fixed tether connector. The remaining free ends of each tether structure 52 may then be deployed around the spinous processes (or attached to a sacrum) in a pattern generally as shown in FIG. 2.

Referring now to FIGS. 5A and 5B, a flexible restraint system 170 will be described. The flexible restraint system 170 includes a sheath having a plurality of battens or wires 172 which reduce interactions between the sheath and restraint system, as well as provide an axial constraint to limit the maximum axial separation of the fixed and adjustable tether connectors 174 and 176, respectively. As shown in FIG. 5A, the battens 172 are axially compressed so that they bow outwardly, distancing the sheath from the tensile member. In FIG. 5B, the fixed and adjustable tether connectors 174 and 176 have moved to their maximum axial separation, straightening the battens 172 and thus limiting further axial separation of the adjustable tether connectors 174 and 176.

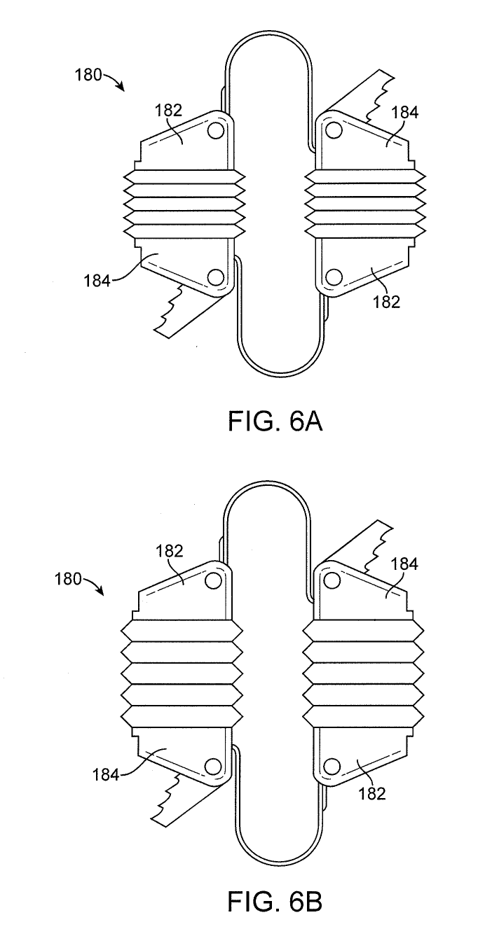

Referring now to FIGS. 6A and 6B, another flexible restraint system 180 constructed in accordance with the principles of the present invention will be described. The flexible restraint system 180 is similar to system 170, except that the sheath structure has an accordion fold to provide for lengthening and shortening together with the movement of fixed and adjustable tether connectors 182 and 184, respectively. The accordion folds both permit greater gross elongation of the sheath with lower material strains than in a purely cylindrical sheath and potentially reduce interaction between the sheath and tensile member. The sheath with the accordion fold may or may not act as a constraint on maximum elongation of the compliance members. The sheath could also be used with separate tension members for providing the maximum elongation limit.

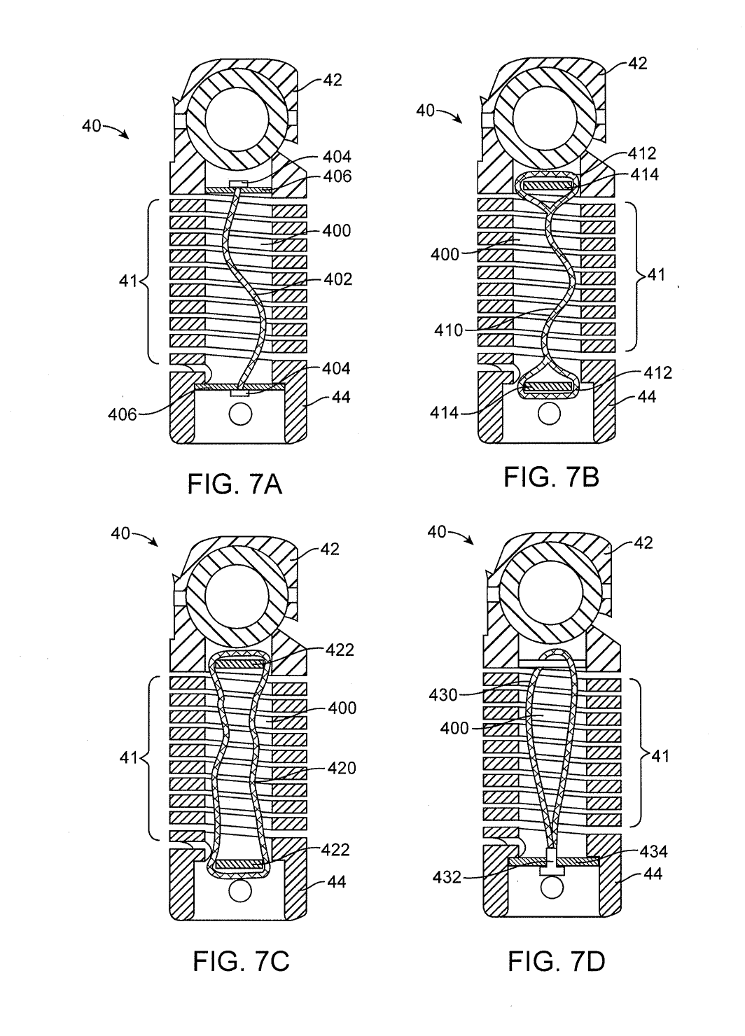

Referring now to FIGS. 7A through 7D, four different embodiments of elongation limit tethers for limiting the maximum elongation of compliance members 40 are illustrated. The compliance members 40 are shown in section with an open chamber 400 shown within the spring structure 41. The open chamber 400 extends between the superior tether attachment element 42 and the inferior tether attachment element 44. As described thus far, the compliance members 40 are identical to those described in FIGS. 3 and 3A above.

A first exemplary tether structure in form of a single cord 402 is illustrated in FIG. 7A. The tether 402 is typically formed from a relatively non-distensible material, such as ultra high molecular weight polyethylene, commercial available under the trade name Dyneema Purity® from suppliers such as DSM®. The cord will be formed so that it is essentially non-distensible, typically with tensile stiffness twice as stiff as the compliance members and preferably ten times as stiff; or typically greater than 20 N/mm, preferably greater than 100 N/mm. The non-distensible cord 402 may have plate, washer or T-shaped anchors 404 at each end and may be held between anchor plates 406 as illustrated. Other mechanisms to fasten a cord or tether to a rigid component may be employed, such as knots, crimps, splices, welds, etc. The inelastic cord 402 will have a certain length of “slack” in it when the compliance member 40 is in its shelf or non-elongated configuration, as shown in FIG. 7A. Thus, the inelastic cord 402 will thus have room to lengthen as the compliance member 40 is stretched when exposed to flexion during use after implantation. The amount of slack will determine the maximum elongation length of the compliance member, typically being in the ranges set forth above.

Referring to FIG. 7B, an alternative non-distensible cord 410 is illustrated, where the cable has loops 412 formed at each end, where the loops may be placed over anchor plates 414. Loops 412 may be preformed or alternatively could be formed using crimps, ties, or other attachments which allow attachment and/or adjustment of length in the field.

Referring now to FIG. 7C, an inelastic cord 420 may comprise a continuous loop which is disposed about upper and lower anchor plates 422. While the continuous loop 420 is illustrated without splices or connection, it would also be possible to provide a connector to allow for adjustment of the length of the loop.

Referring now to FIG. 7D, the inelastic cord may be formed in a single loop 430 attached to a base anchor 432 which is received in an anchor plate 434 in the interior tether attachment 44.

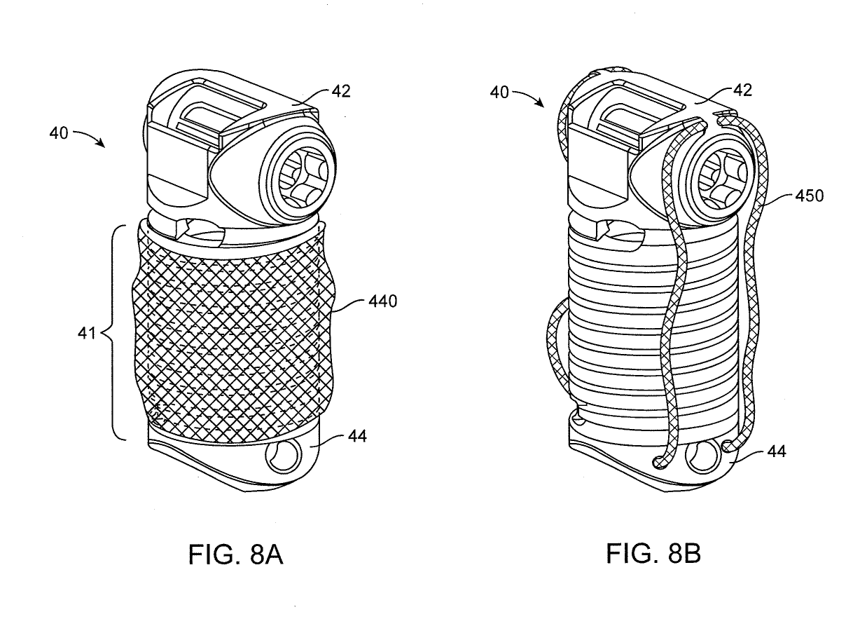

In addition to the internal “cord” tethers of FIG. 7A-7D, the tethers or inelastic constraints may be formed externally over the elastic constraints 40, as illustrated in FIGS. 8A and 8B. For example, the inelastic tether could be a mesh sheath or jacket 440 disposed about the spring structure 41 of the elastic constraint, as shown in FIG. 8A. The mesh sheath 440 would be attached to both the superior tether attachment 42 and inferior tether attachment 44 and would have sufficient slack to allow the desired elongation and maximum elongation limit as the elastic constraint is elongated. Alternatively, as shown in FIG. 8B, the external constraint could be a cable or cord 450 which is threaded through passages in the superior tether attachment 42 and the inferior attachment 44, as illustrated. As with all prior embodiments, the cable or cord 450 would have sufficient slack to allow the desired elongation while providing the hard stop once the desired maximum elongation is reached.

The length of the elongation limit may be set either during fabrication or immediately prior to use. In a first fabrication protocol, the compliance member will be adjusted in a jig or other apparatus to the desired maximum elongation. The tether or inelastic cable or cord which will be used as the elongation limit may then be introduced into or over the elastic constraint and pulled until it is taut. Once it is taut, it can be attached to the anchors, exterior, or otherwise to the body of the compliance member. By attaching when the compliance member is in its desired elongated configuration, the proper relative adjustment of the elongation limit can be assured.

In other instances, however, it may be desirable to adjust the elongation limit in situ after the compliance member has been initially implanted. In such cases, the spinal segment can be manipulated to the desired maximum flexion and the elongation limit fixed to the compliance member while the spinal segment remains in the desired flexion.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A compliance member for elastically constraining spinous processes, said compliance member comprising:
a body having a superior attachment element and an inferior attachment, said body defining a tension spring capable of elastic elongation between said attachment elements, wherein said attachment elements allow the compliance member to be directly or indirectly attached between superior and inferior spinous processes;
an elongation limit coupled between the superior attachment element and the inferior attachment element to prevent elongation of the tension spring beyond a maximum elongation length.
